(19) United States
(12) Patent Application Publication
(10) Pub. No.: US 2025/0241992 A1
(43) Pub. Date: Jul. 31, 2025

(54) COMPOSITIONS AND METHODS FOR TREATING METABOLIC DISEASES

(71) Applicant: Gila Therapeutics, Inc., Minneapolis, MN (US)

(72) Inventors: Andres ACOSTA, Rochester, MN (US); Thomas VASICEK, Minneapolis, MN (US)

(21) Appl. No.: 19/066,477

(22) Filed: Feb. 28, 2025

Related U.S. Application Data
(63) Continuation of application No. 17/730,960, filed on Apr. 27, 2022, now abandoned.
Application No. 17/730,960 is a continuation of application No. 16/243,170, filed on Jan. 9, 2019, now abandoned.
(60) Provisional application No. 62/615,262, filed on Jan. 9, 2018.

(51) Int. Cl. A61K 38/22; A61K 9/00; A61K 9/68; A61K 31/155; A61K 31/366; A61K 31/40; A61K 31/403; A61K 31/4162; A61K 31/495; A61K 31/4985; A61K 31/522; A61K 31/69

(52) U.S. Cl. CPC A61K 38/22; A61K 9/0056; A61K 9/0058; A61K 31/155; A61K 31/366; A61K 31/40; A61K 31/403; A61K 31/4162; A61K 31/495; A61K 31/4985; A61K 31/522; A61K 31/69

(57) ABSTRACT

Compositions comprising satiety peptides (e.g., PYY, PYY(3-36), GLP-1, oxyntomodulin, and cholecystokinin) and DPP-IV inhibitors and methods of treating metabolic diseases with such compositions are provided.

### CROSS-REFERENCE TO RELATED APPLICATIONS

The present application is a Continuation of U.S. patent application Ser. No. 17/730,960 filed on Apr. 27, 2022, which is a Continuation of U.S. patent application Ser. No. 16/243,170 filed on Jan. 9, 2019, which claims priority to and the benefit of U.S. Provisional Application No. 62/615,262 filed Jan. 9, 2018, which is hereby incorporated by reference in its entirety.

All references cited herein, including but not limited to patents and patent applications, are incorporated by reference in their entirety.

### SEQUENCE LISTING

The instant application contains a Sequence Listing which has been submitted electronically in XML format and is hereby incorporated by reference in its entirety. Said XML copy, created on Feb. 26, 2025, is named “51547-005004_Sequence_Listing_2_26_25.xml” and is 3,716 bytes in size.

### BACKGROUND

The prevalence of obesity continues to increase worldwide [1]. In the United States, 69% of adults are overweight or obese [2]. However, there is still a lack of effective, long-term, noninvasive treatments for obesity. The current “one treatment fits all” approach to obesity is associated with highly variable efficacy and outcomes [3].

PYY(3-36) is a Y receptor (e.g., Y2 receptor) agonist released from intestinal cells in response to feeding. Peptide YY (PYY) (3-36) is a satiation gut hormone released postprandially, mainly by the gut. PYY(3-36) secretion is related to caloric intake, and it induces satiation by acting on Y2 receptors in the arcuate nucleus of the hypothalamus. Recently, murine and human PYY(3-36) was found to be present in saliva and its concentration is correlated to its concentration in plasma. PYY(3-36) and Y2 receptors are expressed in the taste cells in the circumvallate papilla of the tongue [4]. In mice, acute augmentation therapy with salivary PYY(3-36) induces higher satiation shown by feeding behavioral studies, and by c-Fos activation in the arcuate nucleus of the hypothalamus. Acute increase of salivary PYY(3-36) resulted in a decrease in one hour food intake in a dose dependent manner. The chronic over-expression of salivary PYY(3-36) using a viral vector-mediated gene delivered (rAAV-PYY vs rAAV-GFP control) into submandibular salivary glands produced a two-fold chronic increase of PYY(3-36) in saliva for 22 weeks [4]. This resulted in a significant decrease in weekly food intake and a 23% body weight loss 8 weeks after vector delivery compared to a control. PYY(3-36) induces satiation through saliva and taste cell receptors [5,6].

Incretins, such as glucagon-like peptide 1 (GLP-1), enhance glycemic control, impede gastric emptying, and increase satiation in healthy and in diabetic patients [7-9]. GLP-1 and GLP-1 agonists reduce fasting and postprandial glucose levels via increased insulin secretion from the pancreas, and reduced gluconeogenesis in the liver.

Exenatide (Exendin-4) is a 39-amino acid peptide that is produced in the salivary gland of the Gila monster lizard. Its amino acid sequence shares 53% identity with GLP-1, but its half-life is prolonged due to its resistance to rapid breakdown by dipeptidyl peptidase 4 (DPP-IV), the normal mechanism for GLP-1 inactivation. Exenatide, in both daily and weekly formulations, has been approved by the FDA for treatment of patients with type 2 diabetes mellitus, where treatment with metformin or sulfonylureas inadequately controls the patient's condition. GLP-1 receptor agonists also retard gastric emptying and decrease food intake by 19% [10-12]. The effects of exenatide on gastric emptying are temporally associated with reduced postprandial glycemia in patients with type 2 diabetes mellitus [13].

DPP-IV inhibitors were developed to increase the circulating levels of endogenous GLP-1, and to treat hyperglycemia. While DPP-IV inhibitors and DPP-IV-resistant GLP-1 receptor agonists have similar effects on glycemia, DPP-IV inhibitors, alone, have no effect on body weight or weight loss. In contrast, GLP-1 receptor agonists have a significant variable effect on weight loss and food intake. For example, studies with GLP-1 receptor agonists have shown an unexplained, highly variable effect on weight loss. Thus, treatment with exenatide, 5 μg SQ twice daily, resulted in weight loss that varied from 2.0±2.8 to 5.1±0.5 kg in 12-24 week studies [14].

Previously, it has been suggested to use DPP-IV inhibitors in combination with weight-loss treatments. However, the DPP-IV inhibitors were to be given after prolonged weight-loss therapy. WO 2011/138421. In addition, oral delivery of weight-loss therapy in combination with DPP-IV inhibitors was not taught or suggested. Id.

### SUMMARY

Aspects described herein provide compositions and methods of treating metabolic disorders (e.g., obesity, diabetes, elevated blood sugar). It has been shown that local oral delivery of PYY(3-36) reduces food intake and increases satiety. See, e.g., U.S. Pat. No. 9,492,505. However, it is desirable to improve the activity of PYY(3-36) with respect to treatment of metabolic disorders. It is also desirable to reduce the dose of PYY(3-36) required to treat metabolic disorders by combining PYY(3-36) with other treatments that provide a combined, additive, or synergistic effect.

In one aspect, compositions comprising PYY(3-36) and a DPP-IV inhibitor are provided, wherein the amount of PYY(3-36) in the composition is no greater than about 250 ng. Further aspects provide pharmaceutical compositions comprising PYY(3-36), a DPP-IV inhibitor, and a pharmaceutically acceptable excipient. In this aspect, the pharmaceutical composition is adapted for local oral delivery.

Yet another aspect provides methods of treating a metabolic disease in a subject by local oral delivery of PYY(3-36) to the subject and administering a DPP-IV inhibitor to the subject.

### DETAILED DESCRIPTION

Before describing an exemplary aspect described herein, it is to be understood that the invention is not limited to the details of construction or process steps set forth in the following description. The aspects described herein are capable of being practiced or being carried out in various ways.

Metabolic diseases or metabolic syndrome refers to diseases that increase risk for diseases associated with, related to, or caused by abnormal metabolism (e.g., diabetes, heart disease, and stroke and include obesity, elevated blood sugar, fatty liver disease, PCOS (polycystic ovary syndrome), and multiple sclerosis). The lack of effective long term, non-invasive procedures for metabolic disorders has spurred the search for small molecules capable of treating these conditions with minimal side effects. While several small molecule therapeutics are currently on the market, their efficacy is relatively low, and safety profiles are less than ideal. On the other hand, natural human hormones responsible for regulation of hunger, satiety and energy metabolism in normal physiology, and their analogs, as described herein, can be used to treat such diseases.

Currently, there is no explanation for the lack of efficacy of DPP-IV inhibitors on weight loss and food intake. However, this lack of efficacy suggests that DPP-IV inhibitors are affecting other food intake pathways. Without being bound by theory, we hypothesize that DPP-IV inhibitors fail to induce weight loss and decrease food intake due to inhibition of the activation of, for example, PYY(1-36) to PYY(3-36). PYY(3-36) is a strong inducer of satiety, while PYY(1-36) is not.

While PYY(3-36) is a strong inducer of satiety, administration of both PYY(3-36) and DPP-IV inhibitors can result in a synergistic increase in satiety, weight loss, and decrease in food intake, as described herein.

GLP-1 receptor agonists, DPP-IV inhibitors, and PYY(3-36) and analogs have been used with limited success in treating metabolic disorders. Therapeutic outcomes with GLP-1 receptor agonists for diabetes mellitus (DM) and obesity are highly variable, and result in significant side effects. While DPP-IV inhibitors have fewer side effects, their use does not appear to induce weight loss, and they are currently indicated exclusively for type 2 DM. Furthermore, systemically administered PYY and analogs tend to be associated with severe side effects, such as nausea and vomiting.

Aspects described herein provide compositions comprising combinations of small molecules (e.g., molecules less than 900 Daltons). In certain aspects, these compositions can be used to treat metabolic diseases (e.g., obesity, diabetes, elevated blood sugar, etc.). The compositions can have additive, synergistic, or increased activity compared to each of the component parts alone. In further aspects, lower doses of each component part of these compositions can be used to reduce, ameliorate, or treat conditions in patients more effectively than untreated patients.

In one aspect, compositions comprising PYY(3-36) and a DPP-IV inhibitor are provided, wherein the amount of PYY(3-36) in the composition is no greater than about 250 ng. In another aspect, the amount of PYY(3-36) in the composition is no greater than about 1 mg or about 10 mg.

In one aspect, the term “PYY(3-36)” or “native PYY-3-36” refers to amino acids 3-36 of the human PYY molecules, having the following amino acid sequence:

(SEQ ID NO: 1)
{NH2}-ILE-LYS-PRO-GLU-ALA-PRO-GLY-GLU-ASP-ALA-SER-
PRO-GLU-GLU-LEU-ASN-ARG-TYR-TYR-ALA-SER-LEU-ARG-
HIS-TYR-LEU-ASN-LEU-VAL-THR-ARG-GLN-ARG-TYR-{COOH).

Native PYY(3-36) is post-translationally processed from a precursor peptide encoded by the following mRNA nucleic acid sequence (positions 632-733 (bolded below)) encoding the mature peptide):

| (SEQ ID NO: 2) | |
|---|---|
| 1 | gcccctggag gaactgaacc cactatcggt catggggccg agactaaatg tggcgggttg |
| 61 | tctttaatct gctgccaaga ggaaactcat tcaggcaagt tcagcccttt atgaggaatt |
| 121 | cccctgtggt cacattccaa ttcctggacc tgctgccacc ctcagaactg catgctcctt |
| 181 | cttcagactt tctaagaatg actcaggtca ttggtggagt gaagtcaaga tttccaactc |
| 241 | agtcacctga agagatggag ataccattca tggagctgga ggtccctgga gatttgggaa |
| 301 | ttcagataac aagctaagat aaggagtttg cctacctctg tcctagagcg aagcctgagc |
| 361 | cttgggcgcg cagcacacca caagtatctg ttactgtgtt ttgcagaagc ttcaggcggg |
| 421 | gatataagcc ccacaaggaa agcgctgagc agaggaggcc tcagcttgac ctgcggcagt |
| 481 | gcagcccttg ggacttccct cgccttccac ctcctgctcg tctgcttcac aagctatcgc |
| 541 | tatggtgttc gtgcgcaggc cgtggcccgc cttgaccaca gtgcttctgg ccctgctcgt |
| 601 | ctgcctaggg gcgctggtcg acgcctaccc catcaaaccc gaggctcccg gcgaagacgc |
| 661 | ctcgccggag gagctgaacc gctactacgc ctccctgcgc cactacctca acctggtcac |
| 721 | ccggcagcgg tatgggaaaa gagacggccc ggacacgctt ctttccaaaa cgttcttccc |
| 781 | cgacggcgag gaccgccccg tcaggtcgcg gtcggagggc ccagacctgt ggtgaggacc |
| 841 | cctgaggcct cctgggagat ctgccaacca cgcccacgtc atttgcatac gcactcccga |
| 901 | ccccagaaac ccggattctg cctcccgacg gcggcgtctg ggcagggttc gggtgcggcc |
| 961 | ctccgcccgc gtctcggtgc ccccgccccc tgggctggag ggctgtgtgt ggtccttccc |
| 1021 | tggtcccaaa ataaagagca aattccacag aaacggaaaa aaaaaaaaa. |

In another aspect, the term “PYY(3-36)” further comprises analogs or variants of native PYY(3-36) that retain at least about 20, 30, 40, 50, 60, 70, 80, 90 or 100% of the biological activity of native PYY(3-36). In this aspect, term “variants” refers to modifications to or substitutions of one or more amino acids of native PYY(3-36). Substitution of an amino acid refers to replacement of one amino acid with another amino acid. In one aspect, an amino acid may be replaced with an amino with a similar side group (e.g., acidic, basic, neutral). The term “biological activity” refers to the activation of Y receptors by one or more small molecules described herein, producing an effect, either locally or systemically, on food intake, gastrointestinal function or central nervous system activity.

Analogs or variants of PYY(3-36) include, for example, the analogs or variants of PYY as described, for example, in U.S. Pat. No. 8,217,001, Michel et al., Dipeptidylpeptidase IV inhibitors in diabetes; more than inhibition of glucagon-like peptide-1 metabolism?Naunyn-Schmiedeberg's Arch Pharmacol (2008) 377:205-207; and Niida et al., Antiobesity and emetic effects of a short-length peptide YY analog and its PEGylated and alkylated derivatives, Bioorganic & Medicinal Chemistry (2017) (S0968-0896) (Epub ahead of print), which are incorporated by reference herein in its entirety. In aspects described herein, PYY(3-36) can be replaced by one or more PYY analogs, or with one or more of the following in place of or in addition to PYY, PYY(3-36), or other PYY analogs: GLP-1, oxyntomodulin, and cholecystokinin acetyl-CoA carboxylase-(ACC) inhibitor, a diacylglycerol O-acyltransferase 1 (DGAT-1) inhibitor, monoacylglycerol O-acyltransferase inhibitors, a phosphodiesterase (PDE)-10 inhibitor, an AMPK activator, a sulfonylurea, a meglitinide, an α-amylase inhibitor, an α-glucoside hydrolase inhibitor, an α-glucosidase inhibitor, a PPARγ agonist, a PPAR α/γ agonist, a biguanide, a glucagon-like peptide 1 (GLP-1) modulator, liraglutide, albiglutide, exenatide, albiglutide, lixisenatide, dulaglutide, semaglutide, a protein tyrosine phosphatase-1B (PTP-1B) inhibitor, SIRT-1 activator, a dipeptidyl peptidase IV (DPP-IV) inhibitor, an insulin secretagogue, a fatty acid oxidation inhibitor, an A2 antagonist, a c-jun amino-terminal kinase (JNK) inhibitor, glucokinase activators (GKa), insulin, an insulin mimetic, a glycogen phosphorylase inhibitor, a VPAC2 receptor agonist, SGLT2 inhibitors, a glucagon receptor modulator, GPR119 modulators, FGF21 derivatives or analogs, TGR5 receptor modulators, GPBAR1 receptor modulators, GPR40 agonists, GPR120 modulators, high affinity nicotinic acid receptor (HM74A) activators, SGLT1 inhibitors, inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostatin receptors, inhibitors or modulators of PDHK2 or PDHK4, inhibitors of MAP4K4, modulators of IL1 family including IL1 beta, HMG-CoA reductase inhibitors, squalene synthetase inhibitors, fibrates, bile acid sequestrants, ACAT inhibitors, MTP inhibitors, lipooxygenase inhibitors, cholesterol absorption inhibitors, PCSK9 modulators, cholesteryl ester transfer protein inhibitors and modulators of RXRα, GIP and GIP agonists, amylin and amylin agonists, ghrelin modulators (e.g., inhibitors) and leptin and leptin agonists, pancreatic polypeptide (PP), calcitonin, OXM, neuropeptide Y (NPY), human growth hormone, prolactin, oxytocin, bovine growth hormone, porcine growth hormone, ghrelin, and glucagon and analogs and variants thereof.

In aspects described herein, the DPP-IV inhibitor is selected from the group consisting of sitagliptin, linagliptin, sitagliptin/metformin, sitagliptin phosphate, linagliptin/metformin, simvastatin, simvastatin/sitagliptin, ildagliptin, saxagliptin, inagliptin, emigliptin, logliptin, relagliptin, marigliptin, omarigliptin, vogliptin, and utogliptin. In another aspect, the term DPP-IV inhibitor refers to a small molecule capable of inhibiting or reducing the activity of dipeptidyl peptidase-IV.

The dosage for the DPP-IV inhibitor can be any suitable dosage based on the condition and patient, for example, from about 2.5 mg to 100 mg depending on the DPP-IV inhibitor. See, e.g., [23-32]. For example, the dose for sitagliptin phosphate can be from about 25-100 mg, the dose for saxagliptin can be from about 2.5-5 mg, the dose for linagliptin can be about 5 mg, the dose for alogliptin can be from about 6.25-25 mg.

Further aspects provide a pharmaceutical composition comprising PYY(3-36) and a DPP-IV inhibitor, and a pharmaceutically acceptable excipient. The term “pharmaceutically acceptable excipient” refers to a non-active ingredient that is accepted or approved for use in human or animal pharmaceutical preparations. In certain aspects, a pharmaceutically acceptable excipient is approved by regulatory authorities for use in human or animal pharmaceuticals.

In another aspect, PYY(3-36) is present in the composition in a concentration of from about 150 picogram pg/ml to about 10 mg/ml, 150 pg/ml to about 5 mg/ml, 150 pg to about 2.5 mg/ml, 150 pg to about 1 mg/ml, and/or 150 pg to about 1 ng/ml. In another aspect, a DPP-IV inhibitor is present in any suitable dose of about 5 mg to 100 mg per day. See, e.g., Deacon et. al., Comparative review of dipeptidylpeptidase-4 inhibitors and sulphonylureas, Diabetes, Obesity and Metabolism 18: 333-347, 2016.

In another aspect, the pharmaceutical composition comprises PYY(3-36), a DPP-IV inhibitor, and a pharmaceutically acceptable excipient. In one aspect, the pharmaceutical composition is adapted for local oral delivery. In another aspect, the pharmaceutical composition comprises a satiety peptide (e.g., GLP-1, oxyntomodulin, and cholecystokinin), a DPP-IV inhibitor, and a pharmaceutically acceptable excipient. The term “adapted for local delivery” refers to a pharmaceutical formulation that can preferentially deliver PYY(3-36) to the oral cavity or, more specifically, the tongue. In another aspect, PYY(3-36) is delivered to the tongue, and binds to a Y receptor (e.g., the Y2 receptor).

The term “binds” refers to an association between PYY(3-36) or a portion of the PYY(3-36) molecule, and a Y receptor through a chemical bond (e.g., ionic, covalent, or hydrophobic) or other chemical or non-chemical association between PYY(3-36) or a portion thereof and a Y receptor, wherein a biological response is induced by the association between PYY(3-36) and the Y receptor. See e.g., Doods, Receptor binding profiles of NPY analogues and fragments in different tissues and cell lines, Peptides. 1995; 16(8):1389-94.

In another aspect, methods of treating a metabolic disease in a subject are provided. In this aspect, the subject can be treated by administering PYY(3-36) (or an analog or variant) and a DPP-IV inhibitor to the subject. In one aspect, PYY(3-36) can be delivered to a subject in need of treatment via local oral delivery. The DPP-IV inhibitor can be administered, for example, at about the same time, sequentially, before, or after PYY(3-36).

In a further aspect, PYY(3-36) is delivered to the tongue. In this aspect, PYY(3-36) can bind to the tongue, and transmit a signal to the brain via a receptor (e.g., Y receptor). In another aspect, PYY(3-36) can be delivered systemically by any suitable route of administration (e.g., oral, parenteral, intravenous, etc.).

In another aspect, the composition further comprises a DPP-IV inhibitor (e.g., sitagliptin, linagliptin, sitagliptin/metformin, sitagliptin phosphate, linagliptin/metformin, simvastatin, simvastatin/sitagliptin, ildagliptin, axagliptin, inagliptin, emigliptin, logliptin, relagliptin, marigliptin, omarigliptin, vogliptin, and utogliptin).

In yet another aspect, the composition further comprises a pharmaceutically acceptable excipient (e.g., diluents, disintegrants, binders, lubricants, glidants, acidifiers, surfactants, gels, creams, foams, pastes, and solvents), wherein the pharmaceutical composition is adapted for local oral delivery (e.g., to the tongue, to a receptor on the tongue (e.g., Y receptor)).

In one aspect the composition can be incorporated in any suitable dosage form (e.g., a lozenge, a dissolvable material, a dissolvable planar sheet, chewing gum, or a solid or semi-solid candy, tablet, orally disintegrating tablet, troche, oral film strip, lyophilized particles, spray-dried particles, etc.).

In another aspect, the composition can be incorporated in a liquid formulation (e.g., emulsion, a syrup, an elixir, a suspension, or a solution). In a further aspect, the composition can be incorporated in a spray for oral administration, or drops for oral administration.

Further aspects provide methods of administering PYY(3-36) (or analogs or variants) to a subject, and administering DPP-IV inhibitor to the subject. In another aspect, the composition can be administered via local oral delivery or systemically to the subject.

In yet another aspect, the PYY(3-36) can be administered to the subject at about the same time as the DPP-IV inhibitor. In another aspect, the PYY(3-36) and DPP-IV inhibitor can be administered together, sequentially, or in any suitable order (e.g., PYY(3-36) before the DPP-IV inhibitor, PYY(3-36) after the DPP-IV inhibitor).

Further aspects provide methods of treating metabolic disease in a subject. In another aspect, the metabolic disease can be selected from the group consisting of obesity, elevated blood sugar, diabetes, fatty liver disease, high blood pressure, PCOS, and multiple sclerosis. In these aspects, “treatment” or “treat” refers to administering or prescribing PYY (e.g., PYY(3-36) or PYY analogue, satiation peptide) and a DPP-IV inhibitor to a patient having the indicated metabolic disease.

Aspects described herein provide compositions comprising PYY(3-36) and a DPP-IV inhibitor, wherein PYY(3-36) is present in a concentration from about 150 pg/ml to about 10 mg/ml, 150 pg/ml to about 5 mg/ml, 150 pg/ml to about 2.5 mg/ml, 150 pg/ml to about 1 mg/ml, and 150 pg/ml to about 1 ng/ml.

In another aspect, the DPP-IV inhibitor is selected from the group consisting of sitagliptin, linagliptin, sitagliptin/metformin, sitagliptin phosphate, linagliptin/metformin, simvastatin, simvastatin/sitagliptin, ildagliptin, saxagliptin, inagliptin, emigliptin, logliptin, relagliptin, marigliptin, omarigliptin, vogliptin, and utogliptin.

Further aspects provide pharmaceutical compositions comprising PYY(3-36), a DPP-IV inhibitor, and a pharmaceutically acceptable excipient, wherein the pharmaceutical composition is adapted for local oral delivery. The term “adapted for local oral delivery” refers to delivery to the oral cavity (e.g., mouth, tongue, and cheek) wherein the delivery of PYY(3-36) to the oral cavity of a subject does not substantially change the concentration of PYY(3-36) in the blood plasma of the subject. In further aspects, the amount of PYY(3-36) in the pharmaceutical compositions is no greater than about 250 ng, 1 mg, or 10 mg. In yet another aspect, the amount of DPP-IV inhibitor in the pharmaceutical composition is from about 2.5 mg to about 100 mg.

In another aspect, the PYY(3-36) in the pharmaceutical composition is delivered to a tongue of the subject. In this aspect, the PYY(3-36) can bind to a receptor on the tongue (e.g., the Y2 receptor).

In yet another aspect, the pharmaceutical composition comprises a lozenge. In this aspect, the lozenge can comprise a dissolvable material. In a further aspect, the lozenge comprises a dissolvable planar sheet, or solid or semi-solid candy. In another aspect, the pharmaceutical composition is in the form of chewing gum.

In another aspect, the composition is a liquid formulation selected from the group consisting of: an emulsion, a syrup, an elixir, a suspension or a solution.

In a further aspect, the liquid formulation is in the form of a spray or drops for oral administration.

Aspects described herein provide methods of treating a metabolic disease in a subject by administering PYY(3-36) to the subject, and administering a DPP-IV inhibitor to the subject. In one aspect, the PYY(3-36) is administered systemically or via local oral delivery to the subject. In a further aspect, the DPP-IV inhibitor is administered systemically or via local oral delivery to the subject.

In another aspect, each of the PYY(3-36) and the DPP-IV inhibitor is administered to the subject at about the same time. In a further aspect, each of the PYY(3-36) and the DPP-IV inhibitor is administered to the subject sequentially. In yet another aspect, the PYY(3-36) is administered to the subject before the DPP-IV inhibitor. In a further aspect, the PYY(3-36) is administered to the subject after the DPP-IV inhibitor.

In one aspect, the DPP-IV inhibitor is selected from the group consisting of sitagliptin, linagliptin, sitagliptin/metformin, sitagliptin phosphate, linagliptin/metformin, simvastatin, simvastatin/sitagliptin, ildagliptin, saxagliptin, inagliptin, emigliptin, logliptin, relagliptin, marigliptin, omarigliptin, vogliptin, and utogliptin.

In a further aspect, the PYY(3-36) is delivered to the tongue of the subject. In another aspect, the PYY(3-36) binds to a receptor on the tongue (e.g., the Y2 receptor).

In another aspect, the metabolic disease is selected from the group consisting of obesity, elevated blood sugar, diabetes, fatty liver disease, PCOS, and multiple sclerosis.

In a further aspect, the metabolic disease is obesity, and food intake by the subject is reduced by about 20% after administering at least one dose of PYY(3-36) and at least one dose of a DPP-IV inhibitor to the subject compared to a subject who did not receive treatment.

In yet another aspect, the metabolic disease is obesity, and the body weight of the subject is reduced by about 5% after administering at least one dose of PYY(3-36) and at least one dose of a DPP-IV inhibitor to the subject compared a subject who did not receive treatment.

In another aspect, the metabolic disease is elevated blood sugar, and blood sugar is reduced by about 10% after administering at least one dose of PYY(3-36) and at least one dose of a DPP-IV inhibitor to the subject compared to a subject who did not receive treatment.

In one aspect, the metabolic disease is diabetes, and the area under the curve in a glucose tolerance test is reduced by about 15% after administering at least one dose of PYY(3-36) and at least one dose of a DPP-IV inhibitor to the subject compared to a subject who did not receive treatment.

In yet another aspect, the metabolic disease is diabetes, and the fasting blood glucose level of the subject is reduced by about 15% after administering at least one dose of PYY(3-36) and at least one dose of a DPP-IV inhibitor to the subject compared to a subject who did not receive treatment.

In one aspect, the metabolic disease is diabetes, and HbA1c levels in the subject are reduced by at least about 15% after administering at least one dose of PYY(3-36) and at least one dose of a DPP-IV inhibitor to the subject compared to a subject who did not receive treatment.

In a further aspect, the metabolic disease is fatty liver disease, and the liver fat concentration is reduced by about 20% after administering at least one dose of PYY(3-36) and at least one dose of a DPP-IV inhibitor to the subject compared to a subject who did not receive treatment.

In yet another aspect, the metabolic disease is PCOS, and wherein PCOS symptoms are reduced by about 15 to 20% after administering at least one dose of PYY(3-36) and at least one dose of a DPP-IV inhibitor to the subject compared to a subject who did not receive treatment.

In a further aspect, wherein the metabolic disease is multiple sclerosis, and multiple sclerosis symptoms are reduced by about 20% after administering at least one dose of PYY(3-36) and at least one dose of a DPP-IV inhibitor to the subject compared to a subject who did not receive treatment.

In another aspect, the metabolic disease is high blood pressure, and the systolic and diastolic blood pressure levels of the subject are reduced by about 20% after administering at least one dose of PYY(3-36) and at least one dose of a DPP-IV inhibitor to the subject compared to a subject who did not receive treatment.

Administering “at least one dose” of an active ingredient refers to administrating a suitable dose for reducing symptoms of the metabolic disease. A suitable dose of PYY(3-36) can include about 250 ng, 1 mg, or 10 mg of PYY(3-36) in a pharmaceutical composition having, for example, a concentration of PYY(3-36) from about 150 pg/ml to about 10 mg/ml, 150 pg/ml to about 5 mg/ml, 150 pg/ml to about 2.5 mg/ml, 150 pg/ml to about 1 mg/ml, and 150 pg/ml to about 1 ng/ml. A suitable dose of a DPP-IV inhibitor can include about 2.5 mg to about 100 mg.

Reducing symptoms associated with a metabolic disease refers to a reduction in symptoms as measured by markers associated with the indicated disease (e.g., as measured by a blood, physical, or genetic test), as self-reported by patients, or as measured in a medical facility or as part of a clinical or other trial.

Further aspects provide pharmaceutical compositions comprising a first active ingredient, a DPP-IV inhibitor, and a pharmaceutically acceptable excipient, wherein the pharmaceutical composition is adapted for local oral delivery.

In this aspect, the first active ingredient is selected from the group consisting of PYY, PYY(3-36), GLP-1, oxyntomodulin, and cholecystokinin, acetyl-CoA carboxylase-(ACC) inhibitor, a diacylglycerol O-acyltransferase 1 (DGAT-1) inhibitor, monoacylglycerol O-acyltransferase inhibitors, a phosphodiesterase (PDE)-10 inhibitor, an AMPK activator, a sulfonylurea, a meglitinide, an α-amylase inhibitor, an α-glucoside hydrolase inhibitor, an α-glucosidase inhibitor, a PPARγ agonist, a PPAR α/γ agonist, a biguanide, a glucagon-like peptide 1 (GLP-1) modulator, liraglutide, albiglutide, exenatide, albiglutide, lixisenatide, dulaglutide, semaglutide, a protein tyrosine phosphatase-1B (PTP-1B) inhibitor, SIRT-1 activator, a dipeptidyl peptidase IV (DPP-IV) inhibitor, an insulin secretagogue, a fatty acid oxidation inhibitor, an A2 antagonist, a c-jun amino-terminal kinase (JNK) inhibitor, glucokinase activators (GKa), insulin, an insulin mimetic, a glycogen phosphorylase inhibitor, a VPAC2 receptor agonist, SGLT2 inhibitors, a glucagon receptor modulator, GPR119 modulators, FGF21 derivatives or analogs, TGR5 receptor modulators, GPBAR1 receptor modulators, GPR40 agonists, GPR120 modulators, high affinity nicotinic acid receptor (HM74A) activators, SGLT1 inhibitors, inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostatin receptors, inhibitors or modulators of PDHK2 or PDHK4, inhibitors of MAP4K4, modulators of IL1 family including IL1 beta, HMG-CoA reductase inhibitors, squalene synthetase inhibitors, fibrates, bile acid sequestrants, ACAT inhibitors, MTP inhibitors, lipooxygenase inhibitors, cholesterol absorption inhibitors, PCSK9 modulators, cholesteryl ester transfer protein inhibitors and modulators of RXRα, GIP and GIP agonists, amylin and amylin agonists, ghrelin modulators (e.g., inhibitors) and leptin and leptin agonists, pancreatic polypeptide (PP), calcitonin, OXM, neuropeptide Y (NPY), human growth hormone, prolactin, oxytocin, bovine growth hormone, porcine growth hormone, ghrelin, and glucagon and analogs and variants thereof.

When the metabolic disease is obesity, food intake by the subject is reduced by at least about 20% after at least one dose of PYY(3-36) and at least one dose of a DPP-IV inhibitor compared to a subject who did not receive treatment. In another aspect, body weight of the subject is reduced by at least about 5% after at least one dose of PYY(3-36) and at least one dose of a DPP-IV inhibitor compared to a subject who did not receive treatment. In the aspects described herein, the term “subject” refers to an animal (e.g., human, non-human) in need of treatment for the indicated disease or condition.

When the metabolic disease is elevated blood sugar (e.g, pre-diabetes), the blood sugar (e.g., glucose) level is reduced by at least about 10% after at least one dose of PYY(3-36) and at least one dose of a DPP-IV inhibitor compared to a subject who did not receive treatment. In another aspect, the fasting blood glucose level is reduced by at least about 10% after at least one dose of PYY(3-36) and at least one dose of a DPP-IV inhibitor compared to a subject who did not receive treatment.

When the metabolic disease is diabetes, the area under the curve (AUC) in a glucose tolerance test is reduced by at least about 15% after at least one dose of PYY(3-36) and at least one dose of a DPP-IV inhibitor compared to a subject who did not receive treatment. In another aspect, HbA1c levels are reduced by at least about 15% after at least one dose of PYY(3-36) and at least one dose of a DPP-IV inhibitor compared to a subject who did not receive treatment.

When the metabolic disease is fatty liver disease, the liver fat concentration is reduced by at least about 20% after at least one dose of PYY(3-36) and at least one dose of a DPP-IV inhibitor compared to a subject who did not receive treatment. In this aspect, liver fat concentration can be measured by, for example, liver biopsy, ultrasound, MRI (magnetic resonance imaging), and elastography.

When the metabolic disease is PCOS, symptoms are reduced by at least about 15 to 20% after at least one dose of PYY(3-36) and at least one dose of a DPP-IV inhibitor compared to a subject who did not receive treatment. In this aspect, the exemplary symptoms include, but are not limited to, hormonal profile (e.g., thyroid function tests, serum prolactin levels, and a free androgen index (defined as total testosterone divided by sex hormone binding globulin [SHBG]×100, to give a calculated free testosterone level), LH2FsH ratio, and testosterone level).

When the metabolic disease is multiple sclerosis, symptoms are reduced by at least about 20% after at least one dose of PYY(3-36) and at least one dose of a DPP-IV inhibitor compared to a subject who did not receive treatment. In this aspect, the symptoms include, but are not limited to, the Multiple Sclerosis Functional Composite. See, e.g., Cutter et al., Development of a multiple sclerosis functional composite as a clinical trial outcome measure, Brain, 1999 May; 122 (Pt 5):871-82.

When the metabolic disease is high blood pressure, systolic and diastolic blood pressure levels are reduced by at least about 20% after at least one dose of PYY(3-36) and at least one dose of a DPP-IV inhibitor compared to a subject who did not receive treatment. In this aspect, for example, treatment can be started when systolic and diastolic blood pressure levels of 140 mm Hg or greater or at diastolic levels of 90 mm are reached.

The term “metabolic disease” refers to a human or animal disease resulting from abnormal function or control of the metabolic system (e.g., obesity, diabetes, fatty liver disease, PCOS, and elevated blood glucose levels).

In another aspect, PYY(3-36) (or an analog or variant) is delivered via local oral delivery to the tongue. Delivery of PYY(3-36) to the tongue minimizes or eliminates any substantial systemic delivery of PYY(3-36). In one aspect, the term “substantial systemic delivery” refers to blood levels of PYY(3-36) or its analogs or variants that exceed the limit of detection, are distinguishable from endogenous levels, or cause a significant change in endogenous levels. In this aspect, PYY(3-36) and DPP-IV inhibitors can be administered for systemic or local oral delivery.

The compositions described herein can be used to treat a patient in need of treatment as described herein. The terms “treat,” “prevent,” or similar terms, as used herein, do not necessarily mean 100% or complete treatment or prevention. Rather, these terms refer to various degrees of treatment or prevention of a particular disease (e.g., 100%, 90%, 80%, 70%, 60%, 50%, 40%, 30%, 20%, 10%, 5%, or 1%) as recognized in the art as being beneficial. The terms “treatment” or “prevention” also refer to delaying onset of a disease for a period of time or delaying onset indefinitely. The term “treatment” or “treating” refers to administering a drug or treatment to a patient or prescribing a drug to a patient where the patient or a third party (e.g., caretaker, family member, or health care professional) administers the drug or treatment.

The components of the compositions described herein also encompass derivatives and analogs. In one embodiment, the terms “derivative” or “analogs” include, but are not limited to, ether derivatives, acid derivatives, amide derivatives, ester derivatives and the like. Methods of preparing these derivatives are known to a person skilled in the art. For example, ether derivatives are prepared by the coupling of the corresponding alcohols. Amide and ester derivatives are prepared from the corresponding carboxylic acid by a reaction with amines and alcohols, respectively.

The components of the compositions described herein also encompass hydrates or solvates of PYY(3-36), DPP-IV inhibitors described herein, and amorphous or crystalline forms (e.g., hemihydrate, monohydrate, dihydrate, trihydrate and the like). Hydrates or solvates of PYY(3-36), DPP-IV inhibitors may be prepared by contacting the compound with water or a solvent under suitable conditions to produce the hydrate or solvate of choice, for example, as described herein.

The compositions described herein also encompass metabolites of the components described herein (e.g., PYY(3-36), DPP-IV inhibitors). “Metabolite” or “metabolites” refer to any substance produced from another substance by metabolism or a through a metabolic process of a living cell or organ.

Any of the components of compositions described herein (e.g., PYY(3-36), DPP-IV inhibitors) can be administered or used as starting materials to be administered orally, parenterally (IV, IM, depot-IM, SQ, and depot-SQ), sublingually, intranasally (inhalation), intrathecally, topically, or rectally. Dosage forms known to those of skill in the art are suitable for delivery of the compositions described herein described herein.

The components of compositions described herein can be formulated into suitable pharmaceutical preparations such as tablets, capsules, or elixirs for oral administration or in sterile solutions or suspensions for parenteral administration. The components of compositions described herein can be formulated into pharmaceutical compositions using techniques and procedures well known in the art.

Any suitable dosage form can be used for delivery of the pharmaceutical compositions described herein. In one aspect, the dosage form is especially suitable for oral delivery. In another aspect, the dosage form is a lozenge (e.g., planar sheet, solid or semi-solid candy). In another aspect, the dosage form is a gel, cream, foam or paste. The lozenge can comprise dissolvable material. In another aspect, the dosage form comprises chewing gum. In yet another aspect, the dosage form is a liquid formulation (e.g., emulsion, syrup, elixir, suspension, or a solution). In a further aspect, the liquid formulation is a spray or drops for oral administration.

In one aspect, about 10 to about 200 mg of the components of compositions described herein, or a physiologically acceptable salt, pro-drug, or co-crystal thereof can be compounded or used as a starting material for compounding with a physiologically acceptable vehicle, carrier, excipient, binder, preservative, stabilizer, flavor, etc., in a unit dosage form as called for by accepted pharmaceutical practice. The amount of active substance in compositions or preparations comprising the components of compositions described herein is such that a suitable dosage in the range indicated is obtained.

In another aspect, the components of compositions described herein can be formulated in a unit dosage form, each dosage containing from about 1 mg to about 1.2 g, or about 2.5 to about 200 mg of each active ingredient. The term “unit dosage from” refers to physically discrete units suitable as unitary dosages for human subjects and other mammals, each unit containing a predetermined quantity of active material calculated to produce the desired therapeutic effect, in association with one or more suitable pharmaceutical excipients.

In one aspect, one or more of the components of compositions described herein are mixed with or used as starting materials mixed with a suitable pharmaceutically acceptable carrier to form compositions. Upon mixing or addition of the compound(s), the resulting mixture may be a solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. In one aspect, the effective concentration is sufficient for lessening or ameliorating at least one symptom of the disease, disorder, or condition treated and may be empirically determined.

Pharmaceutical carriers or vehicles suitable for administration of the components of compositions described herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients (e.g., PYY(3-36) and a DPP-IV inhibitor).

In another aspect, if the components of compositions described herein exhibit insufficient solubility, methods for solubilizing may be used. Such methods are known and include, but are not limited to, using co-solvents such as dimethylsulfoxide (DMSO), using surfactants such as TWEEN, and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

In another aspect, the components of compositions described herein may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

In another aspect, the components of compositions described herein can be enclosed in multiple or single dose containers. The enclosed compounds and compositions can be provided in kits, for example, including component parts that can be assembled for use. For example, one or more of the compounds (e.g., PYY(3-36), DPP-IV inhibitor) can be used as a starting material for a lyophilized form and a suitable diluent may be provided as a separated component for combination prior to use. A kit may include components of compositions described herein and a second or third therapeutic agent for co-administration. The components of compositions described herein and the second or third therapeutic agent may be provided as separate component parts. A kit may include a plurality of containers, each container holding one or more unit dose of the components of compositions described herein. In one aspect, the containers can be adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of the components of compositions described herein will depend on dissolution, absorption, metabolism, and excretion rates of the active compound(s), the dosage schedule, and amount administered as well as other factors known to those of skill in the art.

In another aspect, the active ingredients may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action. The components of compositions described herein can be used, for example, in combination with an anti-obesity, anti-diabetes, or similar drug (e.g., lorcaserin, orlistat, phentermine/topiramate, sibutramine, rimonabant, metformin, exenatide, liraglutide, pamlintide, naltrexone, and tesofensine).

In one aspect, solutions or suspensions used for parenteral, pump delivery, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerin, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA) or its disodium salt; buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropylene glycol, and mixtures thereof. Liposomal suspensions including tissue-targeted liposomes may also be suitable as pharmaceutically acceptable carriers. These may be prepared according to methods known in the art.

In another aspect, the components of compositions described herein may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, hydroxyl propyl methyl cellulose (HPMC), other cellulose derivatives, and the like. Methods for preparation of such formulations are known to those skilled in the art.

In yet another aspect, compounds employed in the methods of the disclosure may be administered enterally or parenterally. When administered orally, compounds employed in the methods of the disclosure can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods described herein need to be administered only once or twice daily.

The oral dosage forms can be administered to the patient 1, 2, 3, or 4 times daily. The components of compositions described herein can be administered either three or fewer times, or even once or twice daily. Whatever oral dosage form is used, it can be designed so as to protect the compounds employed in the methods described herein from the acidic environment of the stomach. Enteric coated tablets and capsules filled with small spheres, each coated to protect from the acidic stomach, are also well known to those skilled in the art and can be used with aspects described herein.

The terms “therapeutically effective amount” and “therapeutically effective period of time” are used to denote treatments at dosages and for periods of time effective to treat, ameliorate, or reduce conditions or symptoms described herein. As noted above, such administration can be parenteral, oral, sublingual, transdermal, topical, intranasal, via a pump, or intrarectal. In one aspect, when administered systemically, the therapeutic composition can be administered at a sufficient dosage to attain a blood level of the compounds of from about 0.001 μM to about 20 μM. For localized administration, much lower concentrations than this can be effective, and much higher concentrations may be tolerated. One of skill in the art will appreciate that such therapeutic effect resulting in a lower effective concentration of the components of compositions described herein may vary considerably depending on the tissue, organ, or the particular animal or patient to be treated. It is also understood that while a patient may be started at one dose, that dose may be varied overtime as the patient's condition changes.

It should be apparent to one skilled in the art that the exact dosage and frequency of administration will depend on the particular compounds employed in the methods of the disclosure administered, the particular condition being treated, the severity of the condition being treated, the age, weight, general physical condition of the particular patient, and other medication the individual may be taking as is well known to administering physicians who are skilled in this art.

### REFERENCES

1. Ng M, Fleming T, Robinson M, et al. Global, regional, and national prevalence of overweight and obesity in children and adults during 1980-2013: a systematic analysis for the Global Burden of Disease Study 2013. Lancet 2014
2. Acosta A, Abu Dayyeh B K, Port J D, Camilleri M. Recent advances in clinical practice challenges and opportunities in the management of obesity. Gut 2014:63:687-695
3. Delgado-Aros S, Kim D-Y, Burton D, et al. Effect of GLP-1 on gastric volume, emptying, maximum volume ingested, and postprandial symptoms in humans. American journal of physiology Gastrointestinal and liver physiology 2002; 282:31
4. van Can J, Sloth B, Jensen C B, et al. Effects of the once-daily GLP-1 analog liraglutide on gastric emptying, glycemic parameters, appetite and energy metabolism in obese, non-diabetic adults. International journal of obesity 2014; 38:784-793
5. Peters A. Incretin-based therapies: review of current clinical trial data. The American journal of medicine 2010; 123:S28-37
6. Verdich C, Flint A, Gutzwiller J P, et al. A meta-analysis of the effect of glucagon-like peptide-1 (7-36) amide on ad libitum energy intake in humans. The Journal of Clinical Endocrinology & Metabolism 2001; 86:4382-4389
7. Bray G, Ryan D. Update on obesity pharmacotherapy. Annals of the New York Academy of Sciences 2014
8. Linnebjerg H, Park S, Kothare P A, et al, Effect of exenatide on gastric emptying and relationship to postprandial glycemia in type 2 diabetes, Regulatory peptides 2008; 151:123-129
9. Moreno J, Willett K, Desilets A. Exenatide as a novel weight loss modality in patients without diabetes. Ann Pharmacother 2012; 46:1700-1706
10. Batterham R L, Cowley M A, Small C J, et al. Gut hormone PYY(3-36) physiologically inhibits food intake. Nature 2002; 418:650-654
11. Flegal K M, Carroll M D, Kit B K, Ogden C L. Prevalence of obesity and trends in the distribution of body mass index among U S adults, 1999-2010. JAMA: the journal of the American Medical Association 2012; 307:491-497
12. Gardiner J V, Jayasena C N, Bloom S R. Gut hormones: a weight off your mind. J Neuroendocrinol 2008:20:834-841
13. Jayasena C N, Bloom S R. Role of Gut Hormones in Obesity. Endocrinology & Metabolism Clinics of North America 2008; 37:769-787
14. Koda S, Date Y, Murakami N, et al. The role of the vagal nerve in peripheral PYY3-36-induced feeding reduction in rats. Endocrinology 2005; 146:2369-2375
15. Le Roux C W, Batterham R L, Aylwin S J, et al. Attenuated peptide Y Y release in obese subjects is associated with reduced satiety. Endocrinology 2006; 147:3-8
16. Lenard N R, Berthoud H-R. Central and Peripheral Regulation of Food Intake and Physical Activity: Pathways and Genes. Obesity 2008; 16:S11-S22
17. Ogden C L, Yanovski S Z, Carroll M D, Flegal K M. The epidemiology of obesity. Gastroenterology 2007:132:2087-2102
18. Green J B et al: Effect of Sitagliptin on Cardiovascular Outcomes in Type 2 Diabetes. N Engl J Med 373:232, 2015.
19. Scirica B M et al: Saxagliptin and cardiovascular outcomes in patients with type 2 diabetes mellitus. N Engl J Med 369:1317, 2013.
20. White W B et al: Alogliptin after acute coronary syndrome in patients with type 2 diabetes. N Engl J Med 369:1327, 2013.
21. Singh S et al: Glucagonlike Peptide 1-Based Therapies and Risk of Hospitalization for Acute Pancreatitis in Type 2 Diabetes Mellitus: A Population-Based Matched Case-Control Study. JAMA Intern Med February 25.
22. Scirica B M et al: The design and rationale of the saxagliptin assessment of vascular outcomes recorded in patients with diabetes mellitus-thrombolysis in myocardial infarction (SAVOR-TIMI) 53 study. Am Heart J 162:818, 2011,
23. Dicker D: DPP-4 inhibitors: impact on glycemic control and cardiovascular risk factors. Diabetes Care 34 Suppl 2:S276, 2011.
24. Rosenstock J et al: Effect of saxagliptin monotherapy in treatment-naïve patients with type 2 diabetes. Curr Med Res Opin 25:2401, 2009.
25. Chia C W, Egan J M: Incretin-based therapies in type 2 diabetes mellitus. J Clin Endocrinol Metab 93:3703, 2008.
28. Nauck M A et al: Efficacy and safety of the dipeptidyl peptidase-4 inhibitor, sitagliptin, compared with the sulfonylurea, glipizide, in patients with type 2 diabetes inadequately controlled on metformin alone: a randomized, double-blind, non-inferiority trial. Diabetes Obes Metab 9:194, 2007.
27. Raz I et al: Efficacy and safety of the dipeptidyl peptidase-4 inhibitor sitagliptin as monotherapy in patients with type 2 diabetes mellitus. Diabetologia 49:2564, 2006.

What is claimed is:

1. A composition comprising PYY(3-36) and a DPP-IV inhibitor, wherein a concentration of PYY(3-36) in the composition is from about 150 pg/ml to about 10 mg/ml.

2. The composition of claim 1, wherein the concentration of PYY(3-36) in the composition is from about 150 pg/ml to about 5 mg/ml.

3. The composition of claim 2, wherein the concentration of PYY(3-36) in the composition is from about 150 pg/ml to about 2.5 mg/ml.

4. The composition of claim 3, wherein the concentration of PYY(3-36) in the composition is from about 150 pg/ml to about 1 mg/ml.

5. The composition of claim 4, wherein the concentration of PYY(3-36) in the composition is from about 150 pg/ml to about 1 ng/ml.

6. The composition of claim 1, wherein the DPP-IV inhibitor is selected from the group consisting of sitagliptin, linagliptin, sitagliptin/metformin, sitagliptin phosphate, linagliptin/metformin, simvastatin, simvastatin/sitagliptin, ildagliptin, saxagliptin, inagliptin, emigliptin, logliptin, relagliptin, marigliptin, omarigliptin, vogliptin, and utogliptin.

7. A pharmaceutical composition comprising PYY(3-36), a DPP-IV inhibitor, and a pharmaceutically acceptable excipient, wherein the pharmaceutical composition is adapted for local oral delivery to a subject.

8. The pharmaceutical composition of claim 7, wherein an amount of PYY(3-36) in the pharmaceutical composition is no greater than about 250 ng.

9. The pharmaceutical composition of claim 7, wherein the amount of PYY(3-36) in the pharmaceutical composition is no greater than about 1 mg.

10. The pharmaceutical composition of claim 7, wherein the amount of PYY(3-36) in the pharmaceutical composition is no greater than about 10 mg.

11. The pharmaceutical composition of claim 7, wherein an amount of DPP-IV inhibitor in the pharmaceutical composition is from about 2.5 mg to about 100 mg.

12. The pharmaceutical composition of claim 7, wherein the PYY(3-36) in the pharmaceutical composition is delivered to a tongue of the subject.

13. The pharmaceutical composition of claim 12, wherein the PYY(3-36) binds to a receptor on the tongue.

14. The pharmaceutical composition of claim 13, wherein the receptor is the Y2 receptor.

15. The pharmaceutical composition of claim 7, wherein the pharmaceutical composition comprises a lozenge.

16. The pharmaceutical composition of claim 15, wherein the lozenge comprises a dissolvable material.

17. The pharmaceutical composition of claim 16, wherein the lozenge comprises a dissolvable planar sheet, or a solid or semi-solid candy.

18. The pharmaceutical composition of claim 7, wherein the pharmaceutical composition is in a dosage form of chewing gum.

19. The pharmaceutical composition of claim 7, wherein the pharmaceutical composition is a liquid formulation selected from the group consisting of an emulsion, a syrup, an elixir, a suspension or a solution.

20. The pharmaceutical composition of claim 19, wherein the liquid formulation is in a dosage form of a spray for oral administration.

21. The pharmaceutical composition of claim 19, wherein the liquid formulation is in a dosage form of drops for oral administration.

22. A method of treating a metabolic disease in a subject comprising, administering PYY(3-36) to the subject, and administering a DPP-IV inhibitor to the subject.

23. The method of claim 22, wherein PYY(3-36) is administered systemically or via local oral delivery to the subject.

24. The method of claim 22, wherein the DPP-IV inhibitor is administered systemically or via local oral delivery to the subject.

25. The method of claim 22, wherein each of the PYY(3-36) and the DPP-IV inhibitor is administered to the subject at about the same time.

26. The method of claim 22, wherein each of the PYY(3-36) and the DPP-IV inhibitor is administered to the subject sequentially.

27. The method of claim 26, wherein the PYY(3-36) is administered to the subject before the DPP-IV inhibitor is administered to the subject.

28. The method of claim 26, wherein the PYY(3-36) is administered to the subject after the DPP-IV inhibitor is administered to the subject.

29. The method of claim 22, wherein the DPP-IV inhibitor is selected from the group consisting of sitagliptin, linagliptin, sitagliptin/metformin, sitagliptin phosphate, linagliptin/metformin, simvastatin, simvastatin/sitagliptin, ildagliptin, saxagliptin, inagliptin, emigliptin, logliptin, relagliptin, marigliptin, omarigliptin, vogliptin, and utogliptin.

30. The method of claim 22, wherein the PYY(3-36) is delivered to a tongue of the subject.

31. The method of claim 30, wherein the PYY(3-36) binds to a receptor on the tongue.

32. The method of claim 31, wherein the receptor is the Y2 receptor.

33. The method of claim 22, wherein the metabolic disease is selected from the group consisting of obesity, elevated blood sugar, diabetes, fatty liver disease, PCOS, and multiple sclerosis.

34. The method of claim 22, wherein the metabolic disease is obesity, and wherein food intake by the subject is reduced by about 20% after at least one dose of PYY(3-36) and at least one dose of a DPP-IV inhibitor compared to a subject who did not receive treatment.

35. The method of claim 22, wherein the metabolic disease is obesity, and wherein the body weight of the subject is reduced by about 5% after at least one dose of PYY(3-36) and at least one dose of a DPP-IV inhibitor compared a subject who did not receive treatment.

36. The method of claim 22, wherein the metabolic disease is elevated blood sugar, and wherein the blood sugar level of the subject is reduced by about 10% after at least one dose of PYY(3-36) and at least one dose of a DPP-IV inhibitor compared to a subject who did not receive treatment.

37. The method of claim 22, wherein the metabolic disease is diabetes, and wherein the area under the curve in a glucose tolerance test of the subject is reduced by about 15% after at least one dose of PYY(3-36) and at least one dose of a DPP-IV inhibitor compared to a subject who did not receive treatment.

38. The method of claim 22, wherein the metabolic disease is diabetes, and wherein a fasting glucose level of the subject is reduced by about 15% after at least one dose of PYY(3-36) and at least one dose of a DPP-IV inhibitor compared to a subject who did not receive treatment.

39. The method of claim 22, wherein the metabolic disease is diabetes, and wherein HbA1c levels of the subject are reduced by at least about 15% after at least one dose of PYY(3-36) and at least one dose of a DPP-IV inhibitor compared to a subject who did not receive treatment.

40. The method of claim 22, wherein the metabolic disease is fatty liver disease, and wherein a liver fat concentration of the subject is reduced by about 20% after at least one dose of PYY(3-36) and at least one dose of a DPP-IV inhibitor compared to a subject who did not receive treatment.

41. The method of claim 22, wherein the metabolic disease is PCOS, and wherein PCOS symptoms in the subject are reduced by about 15 to 20% after at least one dose of PYY(3-36) and at least one dose of a DPP-IV inhibitor compared to a subject who did not receive treatment.

42. The method of claim 22, wherein the metabolic disease is multiple sclerosis, and wherein multiple sclerosis symptoms in the subject are reduced by about 20% after at least one dose of PYY(3-36) and at least one dose of a DPP-IV inhibitor compared to a subject who did not receive treatment.

43. The method of claim 22, wherein the metabolic disease is high blood pressure, and wherein systolic and diastolic blood pressure levels in the subject are reduced by about 20% after at least one dose of PYY(3-36) and at least one dose of a DPP-IV inhibitor compared to a subject who did not receive treatment.

44. A pharmaceutical composition comprising a first active ingredient, a DPP-IV inhibitor, and a pharmaceutically acceptable excipient, wherein the pharmaceutical composition is adapted for local oral delivery.

45. The pharmaceutical composition of claim 44, wherein the first active ingredient is selected from the group consisting of PYY, PYY(3-36), GLP-1, oxyntomodulin, and cholecystokinin, acetyl-CoA carboxylase-(ACC) inhibitor, a diacylglycerol O-acyltransferase 1 (DGAT-1) inhibitor, monoacylglycerol O-acyltransferase inhibitors, a phosphodiesterase (PDE)-10 inhibitor, an AMPK activator, a sulfonylurea, a meglitinide, an α-amylase inhibitor, an α-glucoside hydrolase inhibitor, an α-glucosidase inhibitor, a PPARγ agonist, a PPAR α/γ agonist, a biguanide, a glucagon-like peptide 1 (GLP-1) modulator, liraglutide, albiglutide, exenatide, albiglutide, lixisenatide, dulaglutide, semaglutide, a protein tyrosine phosphatase-1B (PTP-1B) inhibitor, SIRT-1 activator, a dipeptidyl peptidase IV (DPP-IV) inhibitor, an insulin secretagogue, a fatty acid oxidation inhibitor, an A2 antagonist, a c-jun amino-terminal kinase (JNK) inhibitor, glucokinase activators (GKa), insulin, an insulin mimetic, a glycogen phosphorylase inhibitor, a VPAC2 receptor agonist, SGLT2 inhibitors, a glucagon receptor modulator, GPR119 modulators, FGF21 derivatives or analogs, TGR5 receptor modulators, GPBAR1 receptor modulators, GPR40 agonists, GPR120 modulators, high affinity nicotinic acid receptor (HM74A) activators, SGLT1 inhibitors, inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostatin receptors, inhibitors or modulators of PDHK2 or PDHK4, inhibitors of MAP4K4, modulators of IL1 family including IL1 beta, HMG-CoA reductase inhibitors, squalene synthetase inhibitors, fibrates, bile acid sequestrants, ACAT inhibitors, MTP inhibitors, lipooxygenase inhibitors, cholesterol absorption inhibitors, PCSK9 modulators, cholesteryl ester transfer protein inhibitors and modulators of RXRα, GIP and GIP agonists, amylin and amylin agonists, ghrelin modulators (e.g., inhibitors) and leptin and leptin agonists, pancreatic polypeptide (PP), calcitonin, OXM, neuropeptide Y (NPY), human growth hormone, prolactin, oxytocin, bovine growth hormone, porcine growth hormone, ghrelin, and glucagon and analogs and variants thereof.


SEQUENCE LISTING (2 sequences)

SEQ ID NO: 1 - protein, 34 aa, Homo sapiens
IKPEAPGEDASPEELNRYYASLRHYLNLVTRQRY

SEQ ID NO: 2 - DNA, 1069 bp, Homo sapiens
  source 1..1069: mol_type = other DNA
gcccctggaggaactgaacccactatcggtcatggggccgagactaaatgtggcgggttgtctttaatctgctgccaagaggaaactcattcaggcaagttcagccctttatgaggaattcccctgtggtcacattccaattcctggacctgctgccaccctcagaactgcatgctccttcttcagactttctaagaatgactcaggtcattggtggagtgaagtcaagatttccaactcagtcacctgaagagatggagataccattcatggagctggaggtccctggagatttgggaattcagataacaagctaagataaggagtttgcctacctctgtcctagagcgaagcctgagccttgggcgcgcagcacaccacaagtatctgttactgtgttttgcagaagcttcaggcggggatataagccccacaaggaaagcgctgagcagaggaggcctcagcttgacctgcggcagtgcagcccttgggacttccctcgccttccacctcctgctcgtctgcttcacaagctatcgctatggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtatgggaaaagagacggcccggacacgcttctttccaaaacgttcttccccgacggcgaggaccgccccgtcaggtcgcggtcggagggcccagacctgtggtgaggacccctgaggcctcctgggagatctgccaaccacgcccacgtcatttgcatacgcactcccgaccccagaaacccggattctgcctcccgacggcggcgtctgggcagggttcgggtgcggccctccgcccgcgtctcggtgcccccgccccctgggctggagggctgtgtgtggtccttccctggtcccaaaataaagagcaaattccacagaaacggaaaaaaaaaaaaa